(19) United States
(12) Patent Application Publication
(10) Pub. No.: US 2026/0000310 A1
(43) Pub. Date: Jan. 1, 2026

(54) SYSTEMS AND METHODS FOR BLOOD PRESSURE MONITORING

(71) Applicant: Becton, Dickinson and Company, Franklin Lakes, NJ (US)

(72) Inventors: Huy Thanh Vu, Westminster, CA (US); Cynthia Cao Tran, Huntington Beach, CA (US); Greg Joseph Dugan, Corona, CA (US); Abdulkader Sudam, Dana Point, CA (US)

(21) Appl. No.: 19/318,159

(22) Filed: Sep. 3, 2025

Related U.S. Application Data
(63) Continuation of application No. PCT/US2024/018117, filed on Mar. 1, 2024.
(60) Provisional application No. 63/488,372, filed on Mar. 3, 2023.

(51) Int. Cl. A61B 5/0225; A61B 5/022

(52) U.S. Cl. CPC A61B 5/02255; A61B 5/02241; A61B 2562/0247

(57) ABSTRACT

Systems and methods for non-invasive blood monitoring devices are described. Electroactive materials and/or mechanical actuators can be used for non-invasive blood monitoring systems and measure blood pressure.

### CROSS-REFERENCE TO RELATED APPLICATIONS

This application is a continuation of International Application No. PCT/US2024/018117, filed Mar. 1, 2024, which claims the benefit of and priority to U.S. Provisional Patent Application No. 63/488,372 entitled “Systems and Methods for Blood Pressure Monitoring” filed Mar. 3, 2023, the disclosures of which are hereby incorporated by reference in their entireties.

### FIELD OF THE DISCLOSURE

The present disclosure relates to systems and methods for non-invasive blood pressure monitoring; more specifically, systems and methods of non-invasive blood pressure monitoring using actuation systems and eliminating the use of pneumatic systems.

### BACKGROUND OF THE DISCLOSURE

A non-invasive blood pressure monitoring system uses a pneumatic system to monitor beat-to-beat blood pressure. The system is a complex combination of pump and pressure controller module to provide accurate measurements. The pneumatic system has an inherent pulsation of pressure signals that has undesirable audible noise from the reciprocating pump which can affect the quality of the patient's sleep and recovery time.

### SUMMARY OF THE DISCLOSURE

Systems and methods for non-invasive and continuous blood pressure monitoring using actuation systems including (but not limited to) mechanical actuation systems and electroactive polymers are described. The non-invasive blood pressure monitoring systems eliminate the use of pneumatic systems.

One example of the invention includes a blood pressure monitoring system, comprising a carrier body configured for mounting on a body part; a signal source and signal detector pair on the carrier body; an electroactive material (EAP) actuator coupled with the carrier body, wherein the electroactive material actuator is configured to at least partially surround the body part; and a pressure sensor coupled with the EAP actuator and configured to measure a pressure applied by the EAP actuator; and one or more cables configured to provide an electrical signal to actuate the EAP actuator receive an electrical signal from the signal source and signal detector pair, and receive a pressure signal from the pressure sensor to measure a blood pressure of the body part.

In another example, a first side of the carrier body is configured for mounting facing the body part; the signal source and signal detector pair are on the first side of the carrier body; and the EAP actuator is conformed on the first side of the carrier body.

In a further example, the electroactive material actuator has a pair of openings such that the pair of openings surround the signal source and signal detector pair; the pressure sensor has a pair of openings aligned with the pair of openings of the electroactive material actuator; and the signal source and signal detector pair, the electroactive material actuator, and the pressure sensor surround the body part and measure a blood pressure of the body part.

In another example, the signal source and signal detector pair comprise a light emitting diode (LED) and photodiode (PD) pair, respectively.

In an additional example, the body part is selected from the group consisting of: a finger, an arm, an ankle, and a leg.

In a further example, the electroactive material actuator comprises at least one electroactive polymer.

In a further yet example, the electroactive polymer is selected from the group consisting of: an electronic electroactive polymer, a dielectric elastomer, an electrostrictive polymer, a liquid-crystal elastomer, a piezoelectric polymer, an ionic electroactive polymer, a conductive polymer, an ionic polymer-metal composite, and an ionic actuator.

In a yet another example, the electroactive polymer is selected from the group consisting of: polyvinylidene fluoride (PVDF), polyvinylidene fluoride-trifluoroethylene (P(VDF-TrFE)), polypyrrole (PPy), and poly(3,4-ethylenedioxythiophene) (PEDOT).

In another further example, the electrical signal applied to the electroactive material actuator is within an optimal voltage range for the electroactive material actuator.

In a yet further example, the pressure sensor comprises a flexiforce sensor.

In a further example again, the pressure sensor is flexible.

In an additional example, the pressure sensor measures a pressure change induced by the electroactive material actuator.

Another example of the invention includes a blood pressure monitoring system, comprising a carrier body with a first end and a second end forming an open end, the carrier body configured to receive a body part therein; a signal source and signal detector pair; a pressure sensor configured to at least partially surround the body part; a housing having a first side at the open end of the carrier body; an electroactive material actuator located on at least one surface of the first side of the cuff such that the electroactive material is in contact with the first end of the carrier body and configured to expand and contract to widen and narrow the open end and thereby adjust a clamping force exerted by the carrier body on the body part.

In another example yet again, a foam positioned within the open end of the carrier body, the foam configured as a cushion.

In an additional example, the signal source and signal detector pair, the electroactive material actuator, and the pressure sensor are coupled to a cable through a control assembly, wherein the cable provides electrical signal to the electroactive material actuator, receives an electrical signal from the signal source and signal detector pair, and receives a pressure signal from the pressure sensor; and the signal source and signal detector pair, the electroactive material actuator, and the pressure sensor surround the body part and measure a blood pressure of the body part.

In a further yet example again, the signal source and signal detector pair comprise a light emitting diode (LED) and photodiode (PD) pair, respectively.

In another example, the body part is selected from the group consisting of: a finger, an arm, an ankle, and a leg.

In an additional example again, the electroactive material actuator comprises at least one electroactive polymer.

In a yet further example, the electroactive polymer is selected from the group consisting of: an electronic electroactive polymer, a dielectric elastomer, an electrostrictive polymer, a liquid-crystal elastomer, a piezoelectric polymer, an ionic electroactive polymer, a conductive polymer, an ionic polymer-metal composite, and an ionic actuator.

In another yet example, the electroactive polymer is selected from the group consisting of: polyvinylidene fluoride (PVDF), polyvinylidene fluoride-trifluoroethylene (P(VDF-TrFE)), polypyrrole (PPy), and poly(3,4-ethylenedioxythiophene) (PEDOT).

In a further yet example, the electrical signal applied to the electroactive material actuator is within an optimal voltage range for the electroactive material actuator.

In yet another example again, the pressure sensor comprises a flexiforce sensor.

In an additional example, the pressure sensor is flexible.

In a further example, the pressure sensor measures a pressure change induced by the electroactive material actuator.

Another example includes a blood pressure monitoring system, comprising: a carrier body having a first end, a second end, and an open end and configured to receive a body part therein; a mechanical actuator for adjusting a clamping force on the body part by the carrier body; a signal source and signal detector pair on the carrier body; and a pressure sensor on the carrier body configured to measure the clamping force.

In a further yet example, a cuff enclosing the open end of the carrier body, wherein the cuff is connected with a mechanical actuator.

In an additional example again, a foam positioned in the open end of the carrier body, wherein as the carrier body expands or shrinks as a result of the actuation of the mechanical actuator and the foam acts as a cushion.

In a further example again, the signal source and signal detector pair, the mechanical actuator, and the pressure sensor are coupled to a cable through a control assembly, wherein the cable provides electrical signal to the mechanical actuator, receives an electrical signal from the signal source and signal detector pair, and receives a pressure signal from the pressure sensor; and the signal source and signal detector pair, the mechanical actuator, and the pressure sensor surround the body part and measure a blood pressure of the body part.

In an additional further example, the signal source and signal detector pair comprise a light emitting diode (LED) and photodiode (PD) pair, respectively.

In a yet further example again, the body part is selected from the group consisting of: a finger, an arm, an ankle, and a leg.

In a further yet example, the pressure sensor comprises a flexiforce sensor.

In an additional example again, the pressure sensor is flexible.

In a yet further example, the pressure sensor measures a pressure change induced by the mechanical actuator.

Additional examples and features are set forth in part in the description that follows, and in part will become apparent to those skilled in the art upon examination of the specification or may be learned by the practice of the disclosed subject matter. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as examples of the invention and should not be construed as a complete recitation of the scope of the invention, wherein:

FIG. 1A illustrates examples of the volume clamp method for non-invasive blood pressure monitoring systems.

FIG. 1B illustrates a physiological calibration method for monitoring and tracking blood pressure.

FIG. 2A-2B illustrate examples of a non-invasive blood pressure monitoring finger cuff system.

FIGS. 3A-3B illustrate examples of a non-invasive blood pressure monitoring finger cuff incorporating electroactive polymers.

FIGS. 4A-4E illustrate examples of a non-invasive blood pressure monitoring finger cuff incorporating electroactive polymers.

FIGS. 5A-5D illustrate examples of a non-invasive blood pressure monitoring finger cuff incorporating mechanical actuators.

### DETAILED DESCRIPTION OF THE DISCLOSURE

Turning now to the drawings, various systems and methods for non-invasive blood pressure monitoring devices are described. Various blood pressure monitoring systems of the current disclosure eliminate the use of pneumatic pressure systems including (but not limited to) the use of a pneumatic pump in connection with an inflatable bladder or an inflatable cuff. Many of the blood pressure systems described herein implement electroactive polymer (EAP) actuators and/or mechanical actuators within a cuff, to provide pressure for determining blood pressure. The actuators can expand and/or shrink under an applied voltage and produce a desired clamping force to obtain blood pressure measurements. The applied voltage can produce and/or control the clamping force via actuation. In some implementations, flexible pressure sensors are incorporated with the actuators to measure the pressure change induced by actuation. In many implementations, the blood pressure monitoring systems can be placed on various parts of body including (but not limited to) fingers, arms, legs, and ankles, in order to measure an arterial pressure therein. In a number of instances, the non-invasive blood pressure sensors can be incorporated in finger cuffs, arm cuffs, leg cuffs, and ankle cuffs.

The actuator-based non-invasive blood pressure monitoring systems can use the volume clamp method to continuously measure blood pressure. The volume clamp method involves clamping the artery to a constant volume by dynamically providing equal pressure on either side of the arterial wall. The volume can be measured by a photoplethysmograph built into the cuff. The counter pressure can be applied by actuation caused by EAPs or the mechanical actuators. The non-invasive blood pressure monitoring system can continuously record the cuff pressure using pressure sensors to generate real-time arterial pressure waveform.

In several instances, actuators such as electroactive polymers or mechanical actuators can provide actuation to measure and monitor blood pressure. Actuators can exhibit a change in size or shape when stimulated by an electric field. The applied electronic signal can initiate the actuation of the polymer or the mechanical device. Once the electronic signal is removed, the actuators can return to the original size or shape. The amount of actuation can be controlled by the applied electronic signals to apply the force needed to control the diameter of the artery. In some instances, actuators can be connected with pressure sensors to measure the change of forces that are needed to keep arteries in constant volumes in volume clamp methods. The pressure waves can be converted to values of the blood pressure. Signal sources including (but not limited to) light emitters and signal detectors including (but not limited to) light receivers can be placed inside the cuff to measure the volume using a photoplethysmography.

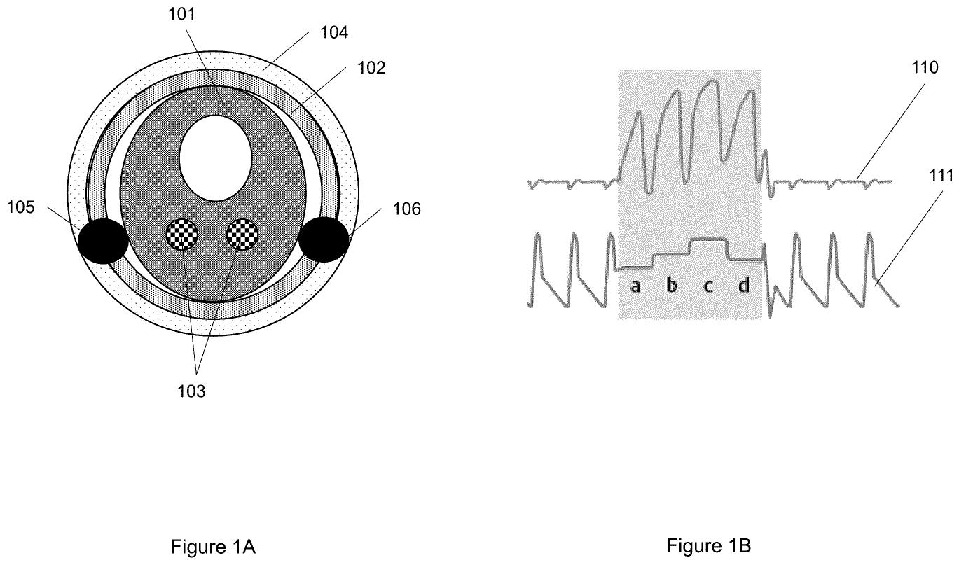

Various non-invasive blood pressure monitoring systems of the current disclosure can use desired mechanisms to measure blood pressure. Some examples of the non-invasive blood pressure monitoring systems use the volume clamp method (also known as vascular unloading technique) to continuously measure blood pressure. FIG. 1A illustrates a schematic of the volume clamp method. A finger cuff can surround the finger. 101 conceptually illustrates a cross section of a finger. The finger cuff is expandable due to the EAPs and/or mechanical actuators. The EAPs and/or mechanical actuators are directly connected with a flexible pressure sensor 102. In some instances, the polymer actuators can overlay with the pressure sensor 102. In several instances, only a portion of the polymer actuators or the mechanical actuators can be connected with the pressure sensor 102. The flexible pressure sensor 102 can be wrapped around with a cuff body 104. The cuff body 104 can be flexible and expandable. As the actuator actuates under an applied electrical signal, the flexible pressure sensor 102 and the cuff body 104 can expand or shrink with the actuator. The finger cuff measures the diameter of the finger artery 103 with an integrated infrared transmission plethysmograph. The integrated plethysmograph can include a light source and/or a light emitter 105 and a light detector and/or a light receiver 106. The finger cuff clamps the artery 103 to a constant volume by dynamically providing equal pressure on either side of the arterial wall. The counter pressure can be applied by actuation caused by the EAPs or the mechanical actuators connected with or positioned inside the cuff body 104. The actuation can be adjusted to desired rate to keep the arterial volume constant. In some instances, high-frequent adjusts of the cuff pressure may be needed to keep the blood volume in the finger artery constant throughout the cardiac cycle.

From the pressure adjustments needed to maintain a constant blood volume in the finger artery, the arterial blood pressure waveform can be derived and analyzed to estimate arterial blood pressure and cardiac output. Based on the counter pressure applied by the actuators and measured by the pressure sensors, and the volume change of the cuff measured by the plethysmograph, the finger arterial pressure waveform can be determined.

Blood pressure monitoring systems can utilize polymer-based actuators within a pressure cuff for providing a clamping force therein. In several examples, electroactive polymers (EAPs) can be utilized in non-invasive blood pressure monitoring systems including (but not limited to) finger cuffs by expanding and/or shrinking under an applied voltage producing the necessary clamping force to obtain blood pressure measurements from finger cuff. The applied electronic power can produce and/or control the clamping force via EAPs actuation. In some examples, mechanical actuation can be incorporated in non-invasive blood pressure monitoring systems including (but not limited to) finger cuffs to provide the clamping force on the finger cuff to attain the blood pressure measurements from finger cuff.

The blood pressure monitoring systems in accordance with several examples can be placed on fingers as finger cuffs. In a number of implementations, the blood pressure monitoring systems can be placed on arms as arm cuffs, or on legs as leg cuffs, or on ankles as ankle cuffs, or on toes as toe cuffs, or on thumb as thumb cuffs.

The non-invasive blood pressure monitoring systems in accordance with several examples herein can use the volume clamp method to continuously measure blood pressure. Calibration may be needed initially and/or frequently to measure the blood pressure. For example, the volume clamp method can be used in conjunction with a physical physiological calibration method for determining an unloaded volume of the arterial wall, as shown in FIG. 1B. The volume clamp method involves clamping the artery to a constant volume by dynamically providing equal pressure on either side of the arterial wall. The volume is measured by a photoplethysmograph built into the cuff. The counter pressure can be applied by actuation caused by EAPs or the mechanical actuators inside the cuff. The use of EAPs or the mechanical actuators can eliminate the inflatable bladder that is common for non-invasive blood pressure. The actuation can be adjusted to desired rate to keep the arterial volume constant. The actuation frequency can be about 200 Hz. The non-invasive blood pressure monitor can continuously record the cuff pressure using pressure sensors to generate real-time finger pressure waveform.

In several instances, electroactive polymers can be used to provide actuation to measure and monitor blood pressure. EAPs can exhibit a change in size or shape when stimulated by an electric field. The applied electronic signal can initiate the actuation of the EAPs. Once the electronic signal is removed, the EAPs can return to the original size or shape. The actuation of EAP can be controlled by the applied electronic signals. The blood pressure monitoring systems in accordance with many examples can use volume clamp mechanism. The actuation can be controlled with a feedback loop to apply the force needed to measure blood pressure. In some instances, EAPs can be connected with pressure sensors to measure the blood pressure. At least a portion of the EAPs can be in direct contact with the pressure sensors such that the pressure sensors can measure the pressure change due to the EAP actuation. In some instances, the EAPs and the pressure can overlap and form a circular shape to wrap around the measurement location. In a number of instances, the pressure censor can be placed inside a cuff. The actuation from EAPs can expand or shrink the cuff. Light emitters and receivers can be placed inside the cuff to measure the volume using a photoplethysmography.

Various types of EAPs can be suitable for non-invasive blood pressure monitoring systems. The EAPs can be used as pure materials or as materials suspended in matrix materials. Matrix materials can comprise polymers. Some examples use piezoelectric and/or ferroelectric EAPs. Examples of EAPs include (but are not limited to): piezoelectric polymers, ferroelectric polymers, electrostrictive polymers, relaxor ferroelectric polymers, dielectric elastomers, liquid crystal elastomers, electrostrictive graft polymers, electrostrictive paper, electrets, electroviscoelastic elastomers and liquid crystal elastomers.

Several instances use PVDF based relaxor ferroelectric based polymers. PVDF based relaxor ferroelectric based polymers show spontaneous electric polarization and they can be pre-strained for improved performance in the strained direction. Examples of PVDF based relaxor ferroelectric based polymers include (but are not limited to): polyvinylidene fluoride (PVDF), polyvinylidene fluoride-trifluoroethylene (PVDF-TrFE), polyvinylidene fluoride-trifluoroethylene-chlorofluoroethylene (PVDF-TrFE-CFE), polyvinylidene fluoride-trifluoroethylene-chlorotrifluoroethylene) (PVDF-TrFE-CTFE), polyvinylidenc fluoride-hexafluoropropylene (PVDF-HFP), polyurethanes or blends thereof.

Some examples use ionic-driven EAPs including (but not limited to): conjugated polymers, carbon nanotube (CNT) polymer composites and ionic polymer metal composites (IPMC).

Many examples use sub-class dielectric elastomers including (but not limited to): acrylates, polyurethanes, silicones. Several examples use sub-class conjugated polymers including (but not limited to): polypyrrole, poly-3,4-ethylenedioxythiophene, poly(p-phenylene sulfide), polyanilines.

In certain instances, mechanical actuators can be used to provide actuation to measure and monitor blood pressure. The mechanical actuators can be powered by DC voltages such as batteries and/or electronic signals. The actuation can be controlled to apply the clamping force needed to measure blood pressure.

Systems and methods for blood pressure monitoring systems in accordance with various examples of the invention are discussed further below.

### Blood Pressure Monitoring Systems

Systems and methods of non-invasive blood pressure monitoring are described. Various non-invasive blood pressure systems can eliminate audible noise, pressure pulsation signals, and/or thermal issues which are by-products of the pneumatic system. The non-invasive blood monitor systems can achieve a compact design. In many examples, the non-invasive blood pressure monitor systems can be incorporated in finger cuffs to continuously monitor blood pressure.

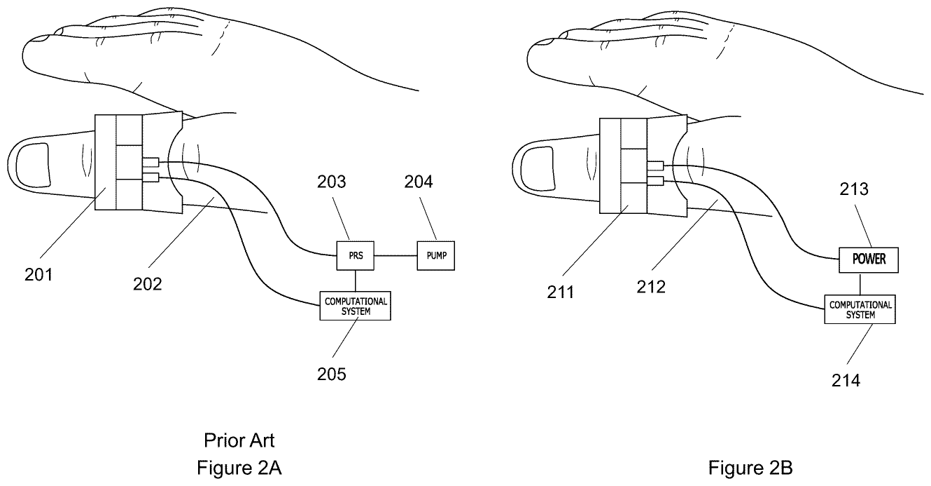

FIG. 2 illustrates a non-invasive blood pressure monitoring system in accordance with an example of the invention. The finger cuff 211 can be positioned around a finger 212 of a patient to continuously monitor the blood pressure. The finger cuff 211 does not require a pressure system to apply the clamp force that is needed to measure blood pressure. The finger cuff 211 can use polymer actuators such as EAPs or mechanical actuators to initiate actuation and apply the required pressure for measurement. The EAPs or the mechanical actuators can be connected with a power source 213. The power source 213 applies a voltage to actuate the EAPs or initiate a mechanical actuation. The EAPs or mechanical actuators can be connected with pressure sensors to actively measure and monitor the clamp force and provide feedback for readout. A computational system 214 can be connected with the finger cuff 211 and the power source 213 to provide feedback and control of the volume change of the actuators.

While various systems for non-invasive blood pressure monitoring are described above with reference to FIG. 2, any variety of systems that utilize actuators to monitor blood pressure can be utilized in the finger cuffs as appropriate to the requirements of specific applications in accordance with various examples of the invention. Systems for EAP finger cuff for blood pressure monitoring in accordance with various examples of the invention are discussed further below.

Blood Pressure Monitoring Systems with Electroactive Polymers

Various systems for non-invasive blood pressure monitoring with electroactive polymers are described. Conventional non-invasive blood pressure monitoring systems use an air compressor diaphragm to provide continuous volume-clamp blood pressure. However, the mechanism of piston and diaphragm produces a pulsation of pressure signals that can have negative effects on the stability and accuracy of the measurement results. The reciprocating pump diaphragm or piston action also produces undesirable audible noise. Excessive noise can affect the quality of the patient's sleep and recovery time. In many instances, EAPs can replace piston and diaphragm (or inflatable bladders or inflatable cuffs) and provide desired pressure required to measure the blood pressure. Several examples use EAPs in finger cuffs for monitoring blood pressure. Integration of EAPs in the finger cuffs can use electronic power to control and produce the clamping force to obtain blood pressure measurements from the finger cuffs. The finger cuff with EAPs can be activated and controlled by electronic power while maintaining steady and precise measurements of system pressure for continuous blood pressure monitoring and measurement. Integration of EAPs in finger cuffs can eliminate audible noise, pressure pulsation signals, and/or thermal issues which can be by-products of the pneumatic systems. Several examples show that the finger cuff with EAPs can achieve a compact design.

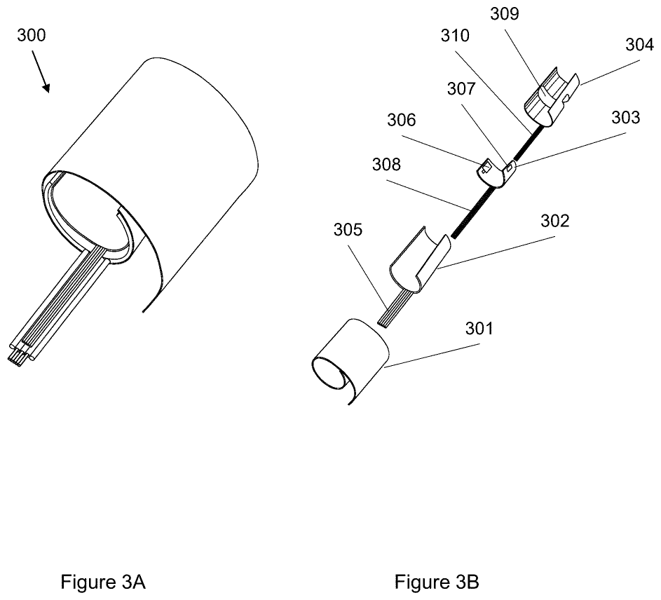

Turing to FIG. 3A, an overview of a finger cuff is illustrated in accordance with an example of the invention. 300 illustrates a finger cuff with EAP replacing the pneumatic system. FIG. 3B illustrates various components of the finger cuff in FIG. 3A in accordance with an example of the invention. The EAP finger cuff 300 can include a cuff body 301, an EAP finger cuff 302, a light emitter and a light receiver sensor 303, and a pressure sensor 304. The cuff body 301 can enclose the EAP finger cuff 302 and wrap the EAP finger cuff 302 around the finger. The EAP replaces the pneumatic system. Various types of EAP can be used as the EAP finger cuff. Examples of EAPs include (but are not limited to) electronic EAPs, ionic EAPs, and ionic actuators. Examples of electronic EAPs include (but are not limited to) dielectric elastomers, electrostrictive polymers, liquid-crystal elastomers (LCEs, typical flexoelectric polymers) and piezoelectric polymers, polyvinylidene fluoride (PVDF), polyvinylidene fluoride-trifluoroethylene (P(VDF-TrFE)). Examples of ionic EAPs include (but are not limited to) conductive polymers, ionic polymer-metal composites (IPMCs), polypyrrole (PPy) and poly(3,4-ethylenedioxythiophene) (PEDOT). Examples of ionic actuators include (but are not limited to) ionic polymer-metal composites. The EAP finger cuff 302 conforms the curvature of the finger and expands to apply pressure when supplied with a voltage. The magnitude of the voltage can be adjusted to the specific polymer used for the EAP finger cuff 302. The supplied voltage can be from about 0 V to about 1 V; or greater than about 1 V and less than about 2 V; or greater than about 2 V. A lead 305 connects the EAP finger cuff 302 with a power source (not shown). The EAP finger cuff 302 can be a single piece of polymer that is fitted inside the cuff body 301. The EAP finger cuff 302 can be a combination of more than one piece of polymer fitter inside the cuff body 301.

A light emitter and a light receiver sensor 303 can be positioned on the EAP finger cuff 302. The light emitter 306 can be positioned cross the finger cuff on the opposite side of the lighter receiver 307. The light emitter can be a light emitting diode (LED). The light receiver can be a photo diode sensor. Any of a variety of LED and photo diode sensor can be utilized in the EAP finger cuff as appropriate to the requirements of specific applications in accordance with various examples of the invention. The LED can have a variety of colors such as green, red, blue, or yellow. Any of a variety of LED color can be utilized as appropriate to the requirements of specific applications in accordance with various examples of the invention. The light emitter and receiver sensor 303 can be connected with a lead 308 with a power supply (not shown).

A pressure sensor 304 can be positioned on top of the EAP finger cuff 302. The pressure sensor 304 conforms the shape of the EAP finger cuff 302 and measures the pressure generated by the EAP finger cuff 302 during operation. The pressure sensor 304 can be a thin and flexible force sensor including (but not limited to) flexiforce sensor. As can readily be appreciated, any of a variety of pressure sensor can be utilized as appropriate to the requirements of specific applications in accordance with various examples of the invention. The pressure sensor 304 can accurately measure the range of pressure for monitoring the blood pressure. The pressure sensor 304 connects with a lead 310 for readout. The pressure sensor 304 can have a cut out 309 where the light emitter and receive sensor 303 can be positioned at.

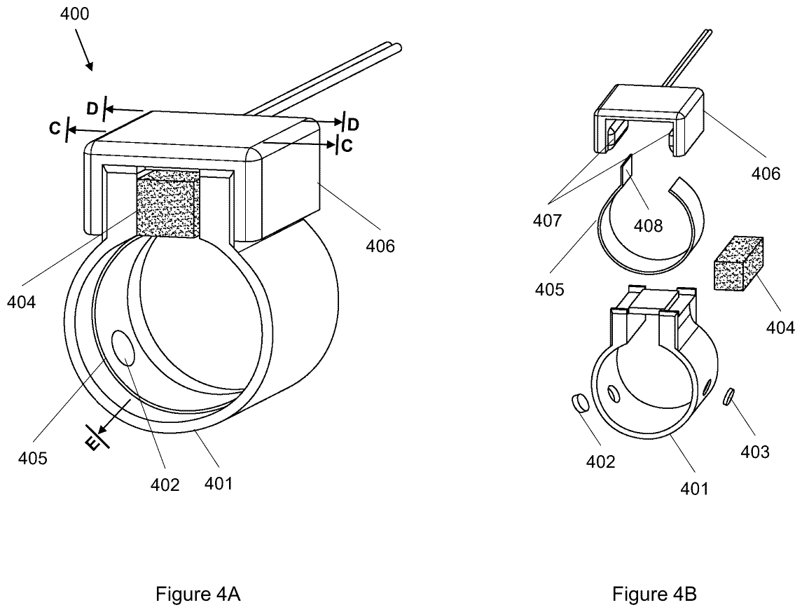

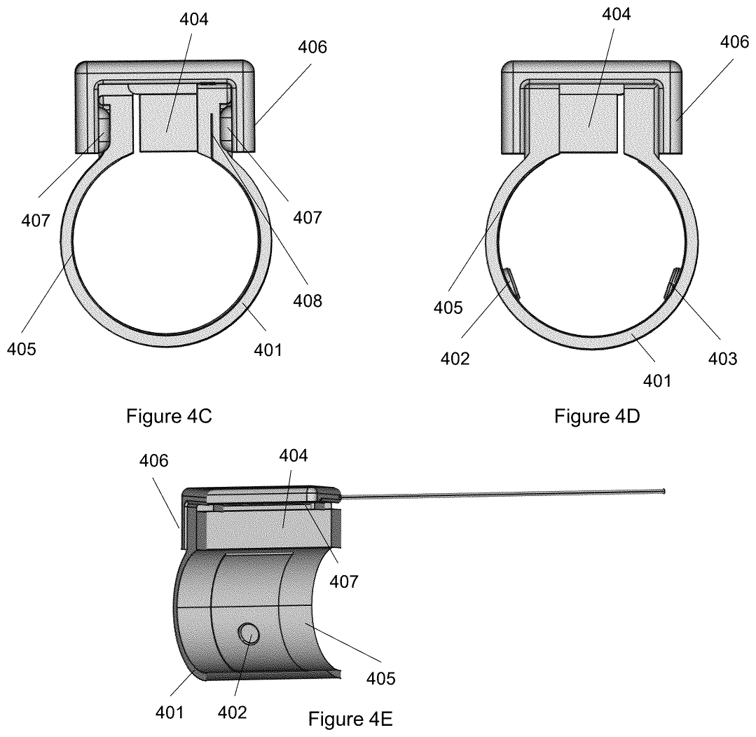

Turning to FIG. 4A, an overview of a finger cuff is illustrated in accordance with an example of the invention. 400 illustrates a finger cuff with EAP replacing the pneumatic system. The finger cuff shown in 400 has a different configuration than the finger cuff shown in 300. FIG. 4B illustrates various components of the finger cuff in FIG. 4A in accordance with an example of the invention. The EAP finger cuff 400 can include a cuff body 401, a light emitter 402, a light receiver 403, a foam (or a sponge) 404, a pressure sensor 405, and an EAP housing 406. The cuff body 401 has a flexible top which can be pushed in or out. A foam 404 can be placed in between the top of the cuff body 401 as shown in FIG. 4A. As the cuff body 401 expand or contract, the foam 404 can act as a buffer. The cuff body 401 can be made with a material including (but not limited to): plastic, polymer, textile, metal, metal alloy, and any combinations thereof. As can be readily appreciated, any of a variety of material can be utilized for the cuff body as appropriate to the requirements of specific applications in accordance with various examples of the invention.

A light emitter 402 and a light receiver 403 can be positioned on the cuff body 401. The light emitter 402 can be facing the light receiver 403. The light emitter and the receiver 403 measure the volume change of the finger artery. The volume change can be converted to pressure of the finger artery. As shown in FIG. 4B, the light emitter 402 and the light receiver 403 are on the opposite side of the cuff body such that the emitting light can transmits through the finger and received by the light receiver. The light receiver 403 is not shown in FIG. 4A. The light emitter can be a LED. The light receiver can be a photo diode sensor. Any of a variety of LED and photo diode sensor can be utilized in the EAP finger cuff as appropriate to the requirements of specific applications in accordance with various examples of the invention. The LED can have a variety of colors such as green, red, blue, or yellow. Any of a variety of LED color can be utilized as appropriate to the requirements of specific applications in accordance with various examples of the invention.

A pressure sensor 405 can be positioned inside the cuff body 401 as shown in FIG. 4A. The pressure sensor 405 can be any type of a force sensor. The pressure sensor 405 can be thin and flexible. The pressure sensor 405 has an extended end 408 on one side. The extended end is in contact with the EAP 407. The extended end 408 is sandwiched between the EAP 407 and the foam 404, such that the pressure sensor 408 can measure the force applied by the EAP 407. The EAP 407 can be positioned on two sides of the EAP housing 406. The EAP housing 406 can apply a voltage to the EAP to control the expansion and shrinkage of the EAP 407. The EAP 407 can apply the required pressure for monitoring blood pressure. The EAP finger cuff 400 can be activated and controlled by the applied voltage to the EAP 407 through the EAP housing 406.

FIG. 4C illustrates a cross section view taken from position C of the EAP finger cuff shown in FIG. 4A in accordance with an example of the invention. As can be seen in the cross section view of FIG. 4C, EAP 407 can be positioned in both sides of the EAP housing 406. The foam 404 is cushioned between the top of the cuff body 401. The pressure sensor 405 is enclosed inside the cuff body 401. The extended end of the pressure sensor 408 is in contact with the EAP 407 to measure the pressure.

FIG. 4D illustrates a cross section view taken from position D of the EAP finger cuff shown in FIG. 4A in accordance with an example of the invention. As can be seen in the cross section view of FIG. 4D, the light emitter 402 and the light receiver 403 can be located on the cuff body 401 and on the pressure sensor 405. The light emitter 402 and the light receiver 403 can be leveled and on opposite sides of the cuff body 401.

FIG. 4E illustrates a cross section view taken from position E of the EAP finger cuff shown in FIG. 4A in accordance with an example of the invention. As can be seen in the cross section view of FIG. 4E, the foam 404 can have the same depth as the cuff body 401 and as the pressure sensor 405.

While various structures for non-invasive EAP blood pressure monitoring systems are described above with reference to FIGS. 3A-3B and FIG. 4A-4E, any variety of finger cuff systems that can be utilized for monitoring blood pressure as appropriate to the requirements of specific applications in accordance with various examples of the invention. Systems for mechanical actuator finger cuff for blood pressure monitoring in accordance with various examples of the invention are discussed further below.

Blood Pressure Monitoring Systems with Mechanical Actuators

Various non-invasive blood pressure monitoring systems with mechanical actuators are described. The non-invasive mechanical actuator blood monitoring systems can eliminate the use of inflatable bladders and/or inflatable cuffs. As a result, the audible noise and pressure pulsation signal from the pneumatic systems can be eliminated. The non-invasive systems have a compact design. The monitoring systems use mechanical actuation to provide the desired pressure to continuously monitor blood pressure. The finger cuff integrating with mechanical actuators can control the mechanical actuation while maintain steady and precise measurements of the blood pressure.

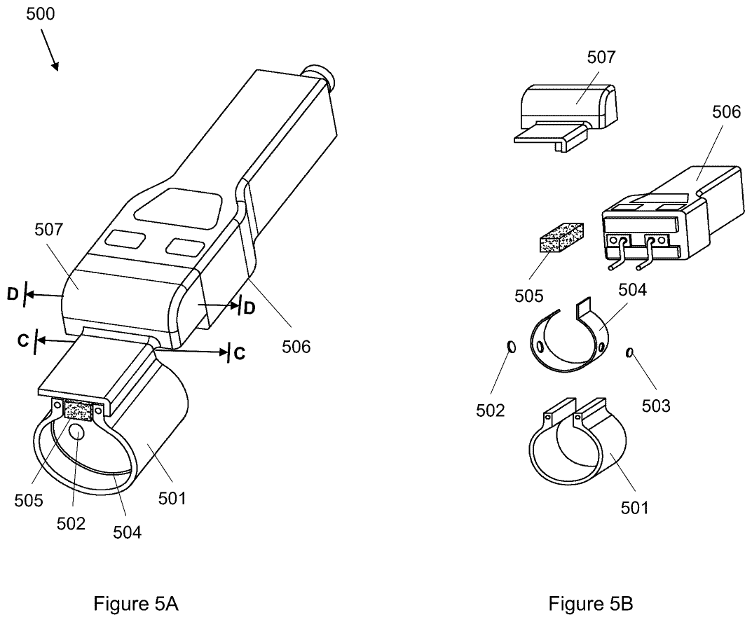

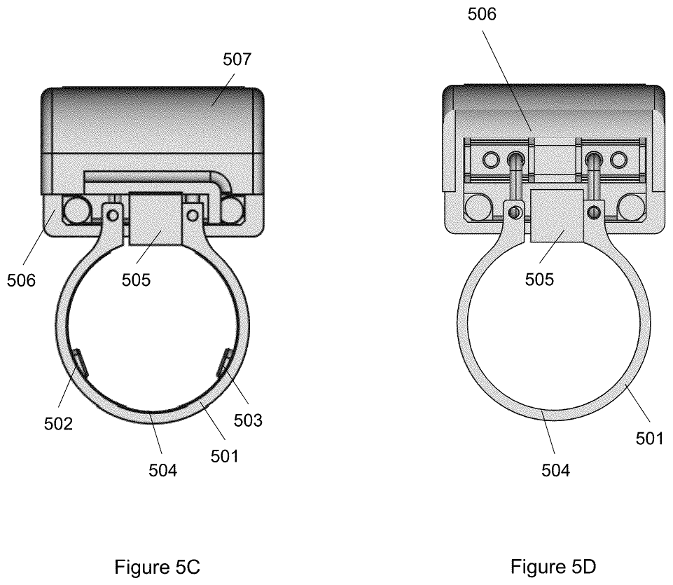

Turing to FIG. 5A, an overview of a finger cuff is illustrated in accordance with an example of the invention. 500 illustrates a finger cuff with a mechanical actuator replacing the pneumatic system. FIG. 5B illustrates various components of the finger cuff in FIG. 5A in accordance with an example of the invention. The mechanical actuator finger cuff 500 can include a cuff body 501, a light emitter 502 and a light receiver 503, a pressure sensor 504, a foam 505, a mechanical actuator 506, and a cover 507. The cuff body 501 can be flexible. As the device actuates to apply a pressure to the finger, the cuff body 501 can expand or shrink. A foam 505 can be positioned on the top of the cuff body 501 as a cushion as the cuff body 501 changes the shape. The pressure sensor 504 can be enclosed inside the cuff body 501. The pressure sensor 504 can measure the applied force by the actuation. A light emitter 502 and a light receiver 503 can be positioned on the pressure sensor 504 and inside the cuff body 501. The light emitter 502 and receiver 503 can be positioned on opposite sides of the finger to measure the volume change of the finger artery. The light emitter can be a LED, and the light receiver can be a photo diode sensor. Any of a variety of LED and photo diode sensor can be utilized in the mechanical actuator finger cuff as appropriate to the requirements of specific applications in accordance with various examples of the invention. The LED can have a variety of colors such as green, red, blue, or yellow. Any of a variety of LED color can be utilized as appropriate to the requirements of specific applications in accordance with various examples of the invention.

A mechanical actuator 506 can be connected with the pressure sensor 504, the foam 505, and the cuff body 501. The mechanical actuator 506 can apply actuation to the cuff body to apply the force required to measure the blood pressure. The mechanical actuator 506 can be powered by a DC signal and/or an AC signal. A cover 507 can be positioned on top of the mechanical actuator 506 to enclose the various parts.

FIG. 5C illustrates a cross section view taken from position C of the mechanical actuator finger cuff shown in FIG. 5A in accordance with an example of the invention. As can be seen in the cross section view of FIG. 5C, mechanical actuator 506 can be positioned in between the cuff body 501 to apply the actuation. The foam 404 is cushioned between the top of the cuff body 401. The pressure sensor 504 is enclosed inside the cuff body 501. The pressure sensor 504 is in contact with the mechanical actuator 506 to measure the pressure. The light emitter 502 and the light receiver 503 can be leveled and on opposite sides of the cuff body 501.

FIG. 5D illustrates a cross section view taken from position D of the mechanical actuator finger cuff shown in FIG. 5A in accordance with an example of the invention. As can be seen in the cross section view of FIG. 5D, the mechanical actuator 506 is in contact with the foam 505 and the pressure sensor 504.

While various structures for the mechanical actuator finger cuff are described above with reference to FIGS. 5A-5D, any variety of structures that utilize mechanical actuators in finger cuff to measure blood pressure can be utilized in the biomedical devices as appropriate to the requirements of specific applications in accordance with various examples of the invention.

### Doctrine of Equivalents

For purposes of this description, certain aspects, advantages, and novel features of the instances of this disclosure are described herein. The disclosed methods, apparatus, and systems should not be construed as being limiting in any way. Instead, the present disclosure is directed toward all novel and nonobvious features and aspects of the various disclosed instances, alone and in various combinations and sub-combinations with one another. The methods, apparatus, and systems are not limited to any specific aspect or feature or combination thereof, nor do the disclosed instances require that any one or more specific advantages be present or problems be solved.

Although the operations of some of the disclosed instances are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Reference to an object in the singular is not intended to mean “one and only one” unless explicitly so stated, but rather “one or more.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

In view of the many possible instances to which the principles of the disclosed technology may be applied, it should be recognized that the illustrated instances are only preferred examples and should not be taken as limiting the scope of the disclosure. Rather, the scope of the disclosure is at least as broad as the following claims.

### EXAMPLES

Example 1: An example of a blood pressure monitoring system, comprising: a carrier body configured for mounting on a body part; a signal source and signal detector pair on the carrier body; an electroactive material (EAP) actuator coupled with the carrier body, wherein the electroactive material actuator is configured to at least partially surround the body part; and a pressure sensor coupled with the EAP actuator and configured to measure a pressure applied by the EAP actuator; and one or more cables configured to provide an electrical signal to actuate the EAP actuator, receive an electrical signal from the signal source and signal detector pair, and receive a pressure signal from the pressure sensor to measure a blood pressure of the body part.
Example 2: The example system of example 1, wherein a first side of the carrier body is configured for mounting facing the body part; the signal source and signal detector pair are on the first side of the carrier body; and the EAP actuator is conformed on the first side of the carrier body.
Example 3: The example system of example 1 or 2, wherein the electroactive material actuator has a pair of openings such that the pair of openings surround the signal source and signal detector pair; the pressure sensor has a pair of openings aligned with the pair of openings of the electroactive material actuator; and the signal source and signal detector pair, the electroactive material actuator, and the pressure sensor surround the body part and measure a blood pressure of the body part.
Example 4: The example system of examples 1, 2, or 3, wherein the signal source and signal detector pair comprise a light emitting diode (LED) and photodiode (PD) pair, respectively.
Example 5: The example system of any one of examples 1-4, wherein the body part is selected from the group consisting of: a finger, an arm, an ankle, and a leg.
Example 6: The example system of any one of examples 1-5, wherein the electroactive material actuator comprises at least one electroactive polymer.
Example 7: The example system of any one of examples 1-6, wherein the electroactive polymer is selected from the group consisting of: an electronic electroactive polymer, a dielectric elastomer, an electrostrictive polymer, a liquid-crystal elastomer, a piezoelectric polymer, an ionic electroactive polymer, a conductive polymer, an ionic polymer-metal composite, and an ionic actuator.
Example 8: The example system of any one of examples 1-7, wherein the electroactive polymer is selected from the group consisting of: polyvinylidene fluoride (PVDF), polyvinylidene fluoride-trifluoroethylene (P(VDF-TrFE)), polypyrrole (PPy), and poly(3,4-ethylenedioxythiophene) (PEDOT).
Example 9: The example system of any one of examples 1-8, wherein the electrical signal applied to the electroactive material actuator is within an optimal voltage range for the electroactive material actuator.
Example 10: The example system of any one of examples 1-9, wherein the pressure sensor comprises a flexiforce sensor.
Example 11: The example system of any one of examples 1-10, wherein the pressure sensor is flexible.
Example 12: The example system of any one of examples 1-11, wherein the pressure sensor measures a pressure change induced by the electroactive material actuator.
Example 13: An example of a blood pressure monitoring system, comprising: a carrier body with a first end and a second end forming an open end, the carrier body configured to receive a body part therein; a signal source and signal detector pair; a pressure sensor configured to at least partially surround the body part; a housing having a first side at the open end of the carrier body; an electroactive material actuator located on at least one surface of the first side of a cuff such that the electroactive material is in contact with the first end of the carrier body and configured to expand and contract to widen and narrow the open end and thereby adjust a clamping force exerted by the carrier body on the body part.
Example 14: The example system of example 13, further comprising: a foam positioned within the open end of the carrier body, the foam configured as a cushion.
Example 15: The example system of example 13 or 14, wherein the signal source and signal detector pair, the electroactive material actuator, and the pressure sensor are coupled to a cable through a control assembly, wherein the cable provides electrical signal to the electroactive material actuator, receives an electrical signal from the signal source and signal detector pair, and receives a pressure signal from the pressure sensor; and wherein the signal source and signal detector pair, the electroactive material actuator, and the pressure sensor surround the body part and measure a blood pressure of the body part.
Example 16: The example system of example 13, 14, or 15, wherein the signal source and signal detector pair comprise a light emitting diode (LED) and photodiode (PD) pair, respectively.
Example 17: The example system of any one of examples 13-16, wherein the body part is selected from the group consisting of: a finger, an arm, an ankle, and a leg.
Example 18: The example system of any one of examples 13-17, wherein the electroactive material actuator comprises at least one electroactive polymer.
Example 19: The example system of any one of examples 13-18, wherein the electroactive polymer is selected from the group consisting of: an electronic electroactive polymer, a dielectric elastomer, an electrostrictive polymer, a liquid-crystal elastomer, a piezoelectric polymer, an ionic electroactive polymer, a conductive polymer, an ionic polymer-metal composite, and an ionic actuator.
Example 20: The example system of any one of examples 13-19, wherein the electroactive polymer is selected from the group consisting of: polyvinylidene fluoride (PVDF), polyvinylidene fluoride-trifluoroethylene (P(VDF-TrFE)), polypyrrole (PPy), and poly(3,4-ethylenedioxythiophene) (PEDOT).
Example 21: The example system of any one of examples 13-20, wherein the electrical signal applied to the electroactive material actuator is within an optimal voltage range for the electroactive material actuator.
Example 22: The example system of any one of examples 13-21, wherein the pressure sensor comprises a flexiforce sensor.
Example 23: The example system of any one of examples 13-22, wherein the pressure sensor is flexible.
Example 24: The example system of any one of examples 13-23, wherein the pressure sensor measures a pressure change induced by the electroactive material actuator.
Example 25: An example of a blood pressure monitoring system, comprising: a carrier body having a first end, a second end, and an open end and configured to receive a body part therein; a mechanical actuator for adjusting a clamping force on the body part by the carrier body; a signal source and signal detector pair on the carrier body; and a pressure sensor on the carrier body configured to measure the clamping force.
Example 26: The example system of example 25, further comprising: a cuff enclosing the open end of the carrier body, wherein the cuff is connected with a mechanical actuator.
Example 27: The example system of example 25 or 26, further comprising: a foam positioned in the open end of the carrier body, wherein as the carrier body expands or shrinks as a result of the actuation of the mechanical actuator and the foam acts as a cushion.
Example 28: The example system of example 25, 26, or 27, wherein the signal source and signal detector pair, the mechanical actuator, and the pressure sensor are coupled to a cable through a control assembly, wherein the cable provides electrical signal to the mechanical actuator, receives an electrical signal from the signal source and signal detector pair, and receives a pressure signal from the pressure sensor; and wherein the signal source and signal detector pair, the mechanical actuator, and the pressure sensor surround the body part and measure a blood pressure of the body part.
Example 29: The example system of any one of examples 25-28, wherein the signal source and signal detector pair comprise a light emitting diode (LED) and photodiode (PD) pair, respectively.
Example 30: The example system of any one of examples 25-29, wherein the body part is selected from the group consisting of: a finger, an arm, an ankle, and a leg.
Example 31: The example system of any one of examples 25-30, wherein the pressure sensor comprises a flexiforce sensor.
Example 32: The example system of any one of examples 25-31, wherein the pressure sensor is flexible.
Example 33: The example system of any one of examples 25-32, wherein the pressure sensor measures a pressure change induced by the mechanical actuator.

1. A blood pressure monitoring system comprising:
a carrier body configured for mounting on a body part;
a signal source and signal detector pair on the carrier body;
an electroactive material (EAP) actuator coupled with the carrier body, wherein the electroactive material actuator is configured to at least partially surround the body part;
a pressure sensor coupled with the EAP actuator and configured to measure a pressure applied by the EAP actuator; and
one or more cables configured to provide an electrical signal to actuate the EAP actuator, receive an electrical signal from the signal source and signal detector pair, and receive a pressure signal from the pressure sensor to measure a blood pressure of the body part.

2. The system of claim 1, wherein:
a first side of the carrier body is configured for mounting facing the body part;
the signal source and signal detector pair are on the first side of the carrier body; and
the EAP actuator is conformed on the first side of the carrier body.

3. The system of claim 2, wherein:
the electroactive material actuator has a pair of openings such that the pair of openings surround the signal source and signal detector pair;
the pressure sensor has a pair of openings aligned with the pair of openings of the electroactive material actuator; and
the signal source and signal detector pair, the electroactive material actuator, and the pressure sensor surround the body part and measure a blood pressure of the body part.

4. The system of claim 1, wherein the signal source and signal detector pair comprise a light emitting diode (LED) and photodiode (PD) pair, respectively.

5. The system of claim 1, wherein the body part is selected from the group consisting of: a finger, an arm, an ankle, and a leg.

6. The system of claim 1, wherein the electroactive material actuator comprises at least one electroactive polymer selected from the group consisting of: an electronic electroactive polymer, a dielectric elastomer, an electrostrictive polymer, a liquid-crystal elastomer, a piezoelectric polymer, an ionic electroactive polymer, a conductive polymer, an ionic polymer-metal composite, and an ionic actuator.

7. The system of claim 1, wherein the electroactive material actuator comprises at least one electroactive polymer selected from the group consisting of: polyvinylidene fluoride (PVDF), polyvinylidene fluoride-trifluoroethylene (P(VDF-TrFE)), polypyrrole (PPy), and poly(3,4-ethylenedioxythiophene) (PEDOT).

8. The system of claim 1, wherein the pressure sensor is flexible.

9. The system of claim 1, wherein the pressure sensor measures a pressure change induced by the electroactive material actuator.

10. A blood pressure monitoring system comprising:
a carrier body with a first end and a second end forming an open end, the carrier body configured to receive a body part therein;
a signal source and signal detector pair;
a pressure sensor configured to at least partially surround the body part;
a housing having a first side at the open end of the carrier body;
an electroactive material actuator located on at least one surface of the first side of a cuff such that the electroactive material is in contact with the first end of the carrier body and configured to expand and contract to widen and narrow the open end and thereby adjust a clamping force exerted by the carrier body on the body part.

11. The system of claim 10, further comprising:
a foam positioned within the open end of the carrier body, the foam configured as a cushion.

12. The system of claim 10:
wherein the signal source and signal detector pair, the electroactive material actuator, and the pressure sensor are coupled to a cable through a control assembly, wherein the cable provides electrical signal to the electroactive material actuator, receives an electrical signal from the signal source and signal detector pair, and receives a pressure signal from the pressure sensor; and
wherein the signal source and signal detector pair, the electroactive material actuator, and the pressure sensor surround the body part and measure a blood pressure of the body part.

13. A blood pressure monitoring system comprising:
a carrier body having a first end, a second end, and an open end and configured to receive a body part therein;
a mechanical actuator for adjusting a clamping force on the body part by the carrier body;
a signal source and signal detector pair on the carrier body; and a
a pressure sensor on the carrier body configured to measure the clamping force.

14. The system of claim 13, further comprising:
a cuff enclosing the open end of the carrier body, wherein the cuff is connected with a mechanical actuator.

15. The system of claim 13, further comprising:
a foam positioned in the open end of the carrier body, wherein as the carrier body expands or shrinks as a result of the actuation of the mechanical actuator and the foam acts as a cushion.

16. The system of claim 13:
wherein the signal source and signal detector pair, the mechanical actuator, and the pressure sensor are coupled to a cable through a control assembly, wherein the cable provides electrical signal to the mechanical actuator, receives an electrical signal from the signal source and signal detector pair, and receives a pressure signal from the pressure sensor; and
wherein the signal source and signal detector pair, the mechanical actuator, and the pressure sensor surround the body part and measure a blood pressure of the body part.

17. The system of claim 13, wherein the signal source and signal detector pair comprise a light emitting diode (LED) and photodiode (PD) pair, respectively.

18. The system of claim 13, wherein the body part is selected from the group consisting of: a finger, an arm, an ankle, and a leg.

19. The system of claim 13, wherein the pressure sensor is flexible.

20. The system of claim 13, wherein the pressure sensor measures a pressure change induced by the mechanical actuator.
